(19) United States
(12) Patent Application Publication
(10) Pub. No.: US 2012/0083647 A1
(43) Pub. Date: Apr. 5, 2012

(54) METHOD FOR CHANGING AN INDIVIDUAL'S STATE OF CONSCIOUSNESS

(76) Inventors: Harry SCHEININ, Piispanristi (FI); Michael ALKIRE, Costa Mesa, CA (US); Kimmo KASKINORO, Raisio (FI); Jaakko LANGSJO, Seinajoki (FI)

(21) Appl. No.: 13/247,102

(22) Filed: Sep. 28, 2011

Related U.S. Application Data
(60) Provisional application No. 61/388,558, filed on Sep. 30, 2010.

(51) Int. Cl. A61N 2/02; A61N 7/00; A61F 7/08; A61N 5/02; A61N 5/067; A61N 5/06

(52) U.S. Cl. 600/13; 607/89; 607/88; 607/96; 607/101; 601/2

(57) ABSTRACT

This invention concerns a method for altering an individual's level of consciousness by directing stimulation or inhibition to a CNS locus target region in the individual's brain that is responsible for consciousness.

### FIELD OF THE INVENTION

This invention concerns a method for altering an individual's state of consciousness by stimulating/or inactivating CNS locus target region/regions in an individual's brain.

### BACKGROUND OF THE INVENTION

The publications and other materials used herein to illuminate the background of the invention, and in particular, cases to provide additional details respecting the practice, are incorporated by reference.

So far, the use of anesthetic drugs is the most important method to affect the state of consciousness in individuals. Anesthetic drugs, such as thiopentone, ketamine, propofol, desflurane, isoflurane, sevoflurane and xenon are used to induce sedation and loss of consciousness (LOC). Also benzodiazepines (e.g. diazepam, lorazepam and midazolam) and alpha2-agonists (e.g. dexmedetomidine) have been used to induce unconsciousness. Furthermore, recovery from sedation and/or anesthesia can be achieved when specific receptor antagonists. E.g. dexmedetomidine-induced effects can be reversed with an alpha2-antagonist atipamezole (Scheinin et al, Anesthesiology 1998; 89:574-584).

Drug-based methods for altering an individual's level of consciousness suffer from many disadvantages: drugs may cause adverse reactions in the individual, they can cause drug dependence (e.g. many sleeping pills), their onset or duration of action is not optimal and their administration may require special techniques and equipment. Furthermore, drugs often have occupational health or environmental disadvantages. E.g. many of the gaseous anesthetics are potent “greenhouse” gases and volatile anesthetics are H-CFC compounds and contribute to the depletion of the ozone layer.

No methods have been published for altering an individual's level of consciousness by noninvasive indirect stimulation of target regions in the individual's brain, where the target regions have been mapped and considered to be responsible for consciousness.

Nexstim Oy, Helsinki, Finland has developed a new method, Nexstim Navigated Brain Stimulation (NBS) System that can be used to create an accurate and detailed map of the critical functions of the cortex using a standard magnetic resonance imaging (MRI) brain scan. This method uses stereotactic MRI-guided transcranial magnetic stimulation (TMS) to non-invasively excite precise areas of the cortex. Sophisticated, real-time data processing and modeling enables unsurpassed accuracy and control of the electric field (E-field) inside the brain. Integrated electromyography (EMG) monitoring instantly shows the responses to stimuli in the central nervous system (CNS) and the peripheral nerves. NBS results are presented as detailed color maps of the critical eloquent areas visualized in a 3-D rendering of the brain. NBS allows confident interpretation of measurement results since it is a direct method, like electrocortical stimulation. This is an important advantage in neurosurgical decision making. This system offers neurosurgeons a safe and painless alternative to direct cortical stimulation that can be performed in comfort prior to surgery. 3-D cortical maps help visualize surgical strategies and give additional information to help patient and physician discuss outcomes.

According to a paper by Desmurget M et al., Science 324, 811-813, 8 May 2009, parietal and premotor cortex regions are serious contenders for bringing motor intentions and motor responses into awareness. The authors used direct electrical stimulation in patients undergoing awake brain surgery. Stimulating the right inferior parietal regions triggered a strong intention and desire to move the contralateral hand, arm or foot, whereas stimulating the left inferior parietal region provoked the intention to move the lips and to talk. When stimulation was increased in parietal areas, the patients believed that they had already performed these movements although no EMG activity was detected. Stimulation of the premotor region triggered overt mouth and contralateral limb movements. Yet, patients denied that they had moved. The study showed that conscious intention and motor awareness arise from increased parietal activity before movement execution.

Massimi et al, Science Vol. 309, 30 Sep. 2005, pp 2228-2232 describe a study to investigate whether changes in cortical information transmission play a role when a person falls asleep and consciousness fades. The authors used TMS together with high-density electroencephalography (EEG) and asked how the activation of one cortical area (the premotor area) is transmitted to the rest of the brain. During quiet wakefulness, an initial response (appr. 15 milliseconds) at the stimulation site was followed by a sequence of waves that moved to connected cortical areas several centimeters away. During non-rapid eye movement sleep, the initial response was stronger but was rapidly extinguished and did not propagate beyond the stimulation site. Thus, the fading of consciousness during certain stages of sleep may be related to a breakdown in cortical effective connectivity.

Ferrarelli F et al., PNAS, Feb. 9, 2010, vol. 107, no. 6, pp 2681-2686, report that by employing TMS in combination with high-density EEG, the cortical effective connectivity is disrupted during early non-rapid eye movement sleep. This is a time when subjects, if awakened, may report little or no conscious content. The authors hypothesized that a similar breakdown of cortical effective connectivity may underlie loss LOC induced by pharmacologic agents. The authors tested the hypothesis by comparing EEG responses to TMS during wakefulness and LOC induced by midazolam. The authors found that wherein TMS triggered responses in multiple cortical areas lasting for >300 ms, during midazolam-induced LOC, TMS-evoked activity was local and of shorter duration. Also this study suggested that a breakdown of cortical effective connectivity may be a common feature of conditions characterized by LOC.

Mark S. George et al., J ECT, Vol. 18, No 4, 2002, pp. 170-181, presents a review of the TMS technique, its therapeutic potential for treating certain neurological and psychiatric diseases, and the combination of TMS with functional imaging and its possible use as a neuroscience tool for study of brain connectivity.

Y Levkovitz et al., Clinical Neurophysiology 118 (2007) 2730-2744 disclose a study using H-coils in TMS research, allowing direct stimulation of deeper neuronal pathways than does standard TMS. The study assessed possible health risks, and some cognitive and emotional effects, of two H-coil versions (H1 and H2) designed to stimulate deep portions of the prefrontal cortex, using several stimulation frequencies. The major finding of the study was that stimulation with the H-coils was well tolerated, with no adverse physical or neurological outcomes. This study provided additional evidence for the feasibility and safety of the two H-coil designs (H1/H2).

The international patent application WO 2008/039930, G Tononi, discloses a method and apparatus for improving the quality of sleep. The person's sensory-motor/parietal cortex brain area is stimulated by a TMS device at less than 5 Hz to promote slow wave sleep EEG activity. Audio, vestibular and nasal stimulation are also mentioned as alternative stimulation methods.

The international patent application WO 2004/006750, discloses a method for inhibiting deception. Functional brain imaging was used to determine the regions of a brain an individual uses to lie or deceive, and then TMS is applied to that region of the brain while an individual is attempting to respond to a directed question about the fact at issue. If the person is attempting to deceive, TMS will temporarily inhibit operation of this part of the brain during this attempted deception, and the individual will be unable to deceive. The anterior cingulate cortex (ACC) is one of the regions of the brain which was found to be significantly involved in the process of deception.

U.S. Pat. No. 6,712,753, Joseph Manne, describes a method and device for sensory stimulation and sensory anesthesia in peripheral nerves. The device creates a time varying magnetic field, which, in turn, creates an electric field in a direction parallel to the nerve and at the nerve so as to cause either depolarization leading to an action potential and subsequent sensory stimulation or hyperpolarization and subsequent blockade of nerve impulses which causes sensory anesthesia. Also brain regions can be targeted. However, the patent does not disclose targeting brain regions that have been considered to be responsible for consciousness.

Thus, none of these documents suggest a concrete method for stimulating or inhibiting a CNS locus target region in an individual's brain that is responsible for consciousness and that could be stimulated directly or through functional connectivity to alter the level of consciousness.

### OBJECTS AND SUMMARY OF THE INVENTION

Based on positron emission tomography (PET) results of a recent study, we have identified deeply located brain structures, such as the ACC, thalamus, hypothalamus, and locus coeruleus (LC) for which the activity seems to be strongly associated with consciousness. The results from this study are disclosed in the Experimental section below and the attached Figures. ACC and other deeply located structures likely play a critical role in consciousness by acting as a key site involved with integrating cognitive-emotional processing (“drive”) with the state of arousal and the intent-to-act (Paus T. Nat Rev Neurosci 2001:2, 417). It has even been suggested that the ACC could be the very site of “free will” in the brain (Crick F. The Astonishing Hypothesis—The Scientific search for the Soul. Scribner, New York, 1994, pp. 265-268).

We therefore believe that stimulating ACC and/or other target areas in the brain will have an effect on an individual's vigilance and level of consciousness.

Thus, the present invention concerns a method for altering an individual's level of consciousness by directing inhibition and/or stimulation to a CNS locus target region/regions in the individual's brain that is/are responsible for consciousness.

### DESCRIPTION OF THE FIGURES

FIG. 1. The drug-dose related pattern of change in regional brain activity associated with various levels of consciousness manipulated with anesthesia as revealed with partial least squares (PLS). Results associated with positive salience of latent variable 1 (LV1) are shown (colored blue to symbolize drug-induced suppressions). (A) Illustration showing the design score pattern in the data that accounts for 68% of the cross block covariance across all 4 scan conditions (LV significant at p<0.0001). (B) Singular image (positive salience) projected onto glass brain three dimensional view of areas significantly associated with this pattern of LV1, shown from a sagittal view (upper image) and from an axial view (lower image) at bootstrap ratio threshold of 3.3 (p<0.001, corrected). (C) Sagittal section (upper image) showing regional effects in posterior cingulate (marker-i) thalamus (marker-ii), precuneus and brainstem areas. Axial section shows posterior cingulate, inferior parietal (marker-iii) and frontal lobe effects. (D) Cortical renderings of inflated surface showing the frontal, parietal (marker-iii) and temporal brain regions associated with LV1. (E) Individual voxel intensity plots of ratio adjusted counts show how selected blue highlighted regions (marked with i, ii, or iii) follow the overall pattern of LV1 (mean±SE) across the study conditions (x,y,z coordinates shown in mm).

FIG. 2. Neural correlates associated with the return of consciousness (ROC) following anesthesia as revealed in the PLS pattern analysis. Results associated with positive salience of latent variable 2 (LV2) are shown (colored red-yellow to symbolize activation). (A) Design score pattern in the data that accounts for 25% of the cross block covariance across all 4 scan conditions (LV significant at p<0.0001). (B) Singular image (positive salience) projected on glass brain three dimensional view of areas significantly associated with this pattern of LV2, shown from a sagittal view (upper image) and from an axial view (lower image) at bootstrap ratio threshold of 3.3 (p<0.001, corrected). (C) Sagittal section (upper image) through thalamus showing regional effects in anterior cingulate (marker-i), thalamus (marker-ii) and brainstem areas (marker-iii). (D) Cortical renderings of inflated surface showing no activations at this threshold. (E) Individual voxel intensity plots of ratio adjusted counts show how selected red highlighted regions (marked with i, ii, or iii) follow the overall pattern of LV2 (mean±SE) across the study conditions.

FIG. 3. Neural correlates of consciousness associated with the ROC and the resumption of the will to move during a steady-state constant dose of dexmedetomidine anesthesia. Results associated with positive salience of latent variable 1 (LV1) are shown (colored red-yellow to symbolize activation). (A) Design score pattern in the data that accounts for 80% of the cross block covariance across all 3 scan conditions (LV significant at p<0.0001). (B) Singular image (positive salience) projected on glass brain three dimensional view of areas significantly associated with this pattern of LV1, shown from a sagittal view (upper image) and from an axial view (lower image) at bootstrap ratio threshold of 3.3 (p<0.001, corrected). (C) Sagittal section (upper image) through thalamus showing regional effects in anterior cingulate (marker-i), thalamus (marker-ii) and brainstem areas. (D) Cortical renderings of inflated surface showing lateral parietal (marker-iii) and frontal cortical activations. (E) Individual voxel intensity plots of ratio adjusted counts show how selected red highlighted regions (marked with i, or iii) follow the overall pattern of LV1 (mean±SE) across the study conditions. Electrical stimulation to the area of the right lateral parietal finding has identified this cortical area as being important in generating the “intention to move” (Desmurget et al., 2009).

FIG. 4. Right lateral parietal cortex (x=52, y=−52, z=46) functional connectivity changes associated with different states of consciousness during a constant dose of dexmedetomidine anesthesia. When conscious, movement intention in the parietal lobe is functionally connected with the intent-to-act in the anterior cingulate (x=−4, y=36, z=20) and other illustrated ventral forebrain regions, but not so in the unconscious condition. (A) Difference image shown on three dimensional glass brain view (sagittal—upper image; axial—lower image) illustrating the brain areas where activity of the parietal cortex is significantly (p<0.05) more correlated in the conscious state as compared with the unconscious state. The seed voxel is shown as a black dot. (B) Cortical projection of the difference image reveals changes in frontal, temporal and occipital areas (yellow circle shows the seed voxel). (C) Sagittal and axial views showing the regional effects in the ventral anterior cingulate, hypothalamus, basal forebrain and anterior cingulate (yellow circle shows region used for correlation plot at bottom of fig). (D) Correlation plot showing the change in functional connectivity relationship between the parietal seed and the anterior cingulate with the change in state of consciousness. Activity in the lateral parietal cortex “intention to move” area is significantly related to activity in the anterior cingulate, but only when subjects are conscious and able to willfully respond. Linear regression lines are shown for conscious (red) and unconscious (blue) states.

FIG. 5. Image showing the correlation between ACC activity and functional connectivity throughout the rest of the brain, independent of the state of consciousness. Areas shown in yellow are negatively correlated with ACC activity in both the conscious and unconscious states. Areas shown in blue are positively correlated with ACC activity in both the conscious and unconscious states. The figure reveals that the activity in Brodmann area (BA) 46 (among a number of others) is highly correlated with ACC activity irrespective of a person's state of consciousness. This means that TMS stimulation of BA 46 should cause an associated change in functional activity within the ACC. Thus, the ACC should be able to be activated or inhibited by the proper TMS stimulus applied to BA 46.

FIG. 6. Image showing the correlations between ACC activity and functional connectivity throughout the rest of the brain that change the most with the changing state of consciousness. Areas shown in yellow are correlated with ACC activity in one state, but not in the other. Areas shown in blue have the opposite functional connectivity pattern with the ACC. The figure reveals that the activity in Brodmann area (BA) 6 (among a few others) is highly correlated with ACC activity depending a person's state of consciousness. This means that TMS stimulation of BA 6 should cause an associated change in functional activity within the ACC, but this might only occur when a person is either conscious, or unconscious. Thus, the ACC should be able to be activated or inhibited by the proper TMS stimulus (excitatory or inhibitory) applied to BA 6, when they are in a conscious or unconscious state.

FIG. 7. An example of MRI diffusion tensor imaging (DTI) (tractography) results from one subject showing a strong anatomical connection between the ACC and BA6 on the right hemisphere.

### DETAILED DESCRIPTION OF THE INVENTION

Using PET in volunteers emerging from anesthesia we compared state-related conditions in which subjects were responsive versus when they were not. The restoration of purposeful movement was associated with increased activity in the arousal and executive control networks, including the reticular formation, hypothalamus, thalamus and the ACC. Restoring consciousness during constant-dose anesthesia revealed activation of the same arousal network and concurrent activation of the parietal cortex intentional movement area. Connectivity analysis of this region further demonstrated that volitional movement can occur when the parietal cortex functionally connects with anterior motor networks. These results imply that anesthesia suppresses the appearance of consciousness by actions on the arousal and motor intention networks.

The method according to the invention is useful, for example, to awaken a subject from anesthesia or sedation using TMS or other stimulation to brain areas known to be important in the control of human consciousness. Such a brain area would be the ACC, but based on our present and previous PET results, and other recent data, also other CNS loci, e.g. the cuneus, precuneus, parietal cortex, prefrontal cortex, thalamus, hypothalamus, brainstem and LC could constitute the targets for local or remote stimulation. The ACC is located relatively deep in the brain (approximately 4 cm from the brain surface) but with special TMS coils (e.g. the H-coil) it is possible to stimulate transcranially these deeper structures. Another possibility is to stimulate more superficial structures, e.g. certain cortical areas in the premotor and parietal cortex, that are functionally connected to deeper brain areas, like the ACC, to induce activation in the latter.

The method could be used to speed up the recovery from anesthesia but also to investigate the arousability of a comatose or heavily sedated patient, e.g., in the intensive care or emergency medicine settings. The neurological state (e.g. using the Glasgow Coma Scale) of a subject could be instantaneously and repeatedly determined with targeted TMS or other stimulation and without the need for dose adjustment of the sedative agent. The response to stimulation could also serve as a surrogate for clinical outcome (prognosis).

It is also possible to deactivate brain activity with certain TMS stimulation frequencies. This opens the possibility to anesthetize or sedate a subject, or even induce normal physiological sleep “remotely”, without an anesthetic, sedative drug or a sleeping pill. The possibility of changing a subject's state of consciousness instantly and non-pharmaceutically could revolutionize medicine and also have an enormous impact on our daily life. Also deactivation could be induced directly to the target brain area or indirectly utilizing cortico-cortical or cortical-subcortical connections.

Depending on the nature of correlation (positive or negative) between the ACC and the target (superficial) cortical structure and state of the subject (awake or unconscious), the effects of inhibitory and excitatory TMS can be used. E.g., if the correlation is negative between the target structure and the ACC, excitatory TMS could theoretically inhibit and inhibitory TMS activate the ACC. Also several target regions could be inhibited and/or stimulated simultaneously.

### DEFINITION OF TERMS

The term “individual” shall include any mammal. Thus both humans and animals are included.

The term “level of consciousness” refers to consciousness and states of decreased consciousness or LOC due to anesthesia, sedation, sleep, coma, vegetative state, and minimally conscious state.

The wording “CNS locus target region/regions in the individual's brain that is/are responsible for consciousness” includes target regions directly responsible for consciousness. Such target regions are, for example the ACC, cuneus, precuneus, prefrontal cortex, thalamus, hypothalamus, brainstem arousal centers (e.g. LC), and the parietal cortex (e.g. the intentional movement area).

“Superficial structures” connected to ACC include, for example prefrontal cortex, cortical areas of motor performance (e.g. the primary motor cortex, the supplementary motor area, the premotor cortex and the parietal cortex intentional movement area) as well as certain regions involved with sensory functions (e.g. auditory cortex areas). Superficial structures connected to other target regions such as cuneus, precuneus, thalamus, hypothalamus, LC, can also be stimulated.

The term “stimulation” includes particularly TMS, but also other forms of stimulation such as audio stimulation, vestibular stimulation and nasal stimulation are included. If target regions are located deeply in the brain, such as ACC, it may be useful to use H-coils or other coils capable of deep stimulation instead of the standard figure-8 TMS coil (Lekovitz, 2007).

The term “determining the target region in an individual's brain” includes non-functional determining of structural (anatomical) features of an individual's brain and/or functional brain mapping methods. However, many of the methods are both anatomical and functional mapping methods. Some examples of these methods for determining the target regions are MRI, diffusion tensor imaging (DTI) MRI, functional MRI (fMRI), magnetic resonance spectroscopy and other MRI based methods, PET, single photon emission computed tomography (SPECT), quantitative EEG (qEEG), magnetoencephalography (MEG), ultrasound (US), near-infrared spectroscopy (NIRS) and other optical methods, computer tomography (CT) and electrical impedance tomography (BIT).

“Navigated stimulation” refers to directing the TMS precisely to regions that have been mapped in advance.

### PREFERRED EMBODIMENTS

According to one embodiment of the invention, a region directly responsible for consciousness is targeted. Such target regions are, for example the ACC, cuneus, precuneus, prefrontal cortex, thalamus, hypothalamus, brainstem arousal areas (e.g. LC), and the parietal cortex (e.g. the intentional movement area). Also several regions can be targeted simultaneously.

If the target region is located deeply in the brain (approximately 4 cm or more from the brain surface) it is preferable to carry out the stimulation and/or inhibition by TMS, particularly by using an H-coil (Roth et al. Clin Neurophysiol 24, 31-38 (2007)). Examples of deeply located target regions are the ACC, thalamus, hypothalamus and brain stem (e.g. LC).

Alternatively, a deeply located target region such as the ACC or other deeply located targets can be indirectly affected by stimulation and/or inhibition of a superficial structure connected to the deeply located target, whereby change in activity is induced in the deeply located target. Such a superficial structure can be a cortical area in the premotor cortex or sensory-motor/parietal cortex, in particular the parietal cortex intentional movement area (Paus et al., Eur J Neurosci 14, 1405-1411 (2001)). Such structures can be identified with, for example, DTI MRI to enable optimal indirect stimulation.

In case a superficial structure shall be stimulated TMS (either standard TMS or TMS based on the use of an H-coil) is suitable. For stimulating superficial structures also other kinds of stimulations may be useful, for example audio stimulation, US, vestibular stimulation, nasal stimulation, focused laser beams, any form of electromagnetic radiation, light, heat and microwave, to make a neuron fire.

In one embodiment the stimulation is used to return the individual's consciousness from a state of anesthesia, sedation, sleep, coma, vegetative state, and minimally conscious state. In this situation excitatory TMS-stimulation using a frequency exceeding 5 Hz is very suitable. Inhibitory (less than 1 Hz) TMS may also be efficacious if correlation (effective connectivity) between the targeted (superficial) and the deep structure (e.g. the ACC) is negative.

Alternatively, the individual's level of consciousness can be decreased, for example by an inhibitory TMS-stimulation using a frequency less than 1 Hz. Accordingly, stimulatory (more than 5 Hz) TMS may be used if correlation (effective connectivity) between the targeted (superficial) and the deep structure (e.g. the ACC) is negative.

Before targeting the individual's brain by stimulation such as TMS, at least one target region in the individual's brain may be determined. Such mapping can be carried out in a separate session before the stimulation or simultaneously with the stimulation. Alternatively, stimulation such as TMS or other stimulation can be applied directly to induce or reverse sedation, sleep or anesthesia without prior brain mapping.

It is particularly preferable to first map the individual's brain by a non-functional method carried out in a separate session before initiating the stimulation. As examples of such non-functional methods can be mentioned MRI or DTI MRI. Alternatively, the mapping can be carried out by obtaining a brain map of the brain with fMRI and other MRI based methods, PET, SPECT, qEEG, MEG, US, NIRS and other optical methods, CT and EIT.

According to a particularly preferred method a target region in the individual's brain is determined by both a non-functional method and additionally by a functional brain mapping method before initiating the stimulation.

The invention will be illuminated by the following non-restrictive Experimental Section.

### EXPERIMENTAL SECTION

### Study I

### Neural Correlates of Consciousness and the Will to Move

Conscious state is clinically assumed when a specific command results in a purposeful movement. Using neuroimaging in volunteers emerging from anesthesia we compared state-related conditions in which subjects were responsive versus those when they were not. The restoration of purposeful movement was associated with increased activity in arousal and executive control networks, including the reticular formation, hypothalamus, thalamus and anterior cingulate. Restoring conscious state during constant-dose anesthesia revealed activation of the same arousal network and concurrent activation of the parietal cortex intentional movement area. Connectivity analysis of this region further demonstrated that willful movement is possible when the parietal cortex functionally connects with anterior motor networks. We conclude that anesthesia suppresses responsiveness by actions on the arousal and motor intention networks.

Anesthesia offers an important tool for the scientific study of consciousness (H. K. Beecher, Science 105, 164 (1947); M. T. Alkire et al. Science 322, 876 (Nov. 7, 2008)). By testing responsiveness to a verbal command in volunteers undergoing the process of anesthesia we sought the neural correlates associated with the change in conscious state by using modern neuroimaging techniques. Whereas a number of human neuroimaging studies have examined brain functioning during the onset of anesthetic-induced unconsciousness (M T Alkire et al., 2008), few have systematically examined the neural correlates associated with the return of the conscious state. This is a critical study design issue because the drug-induced unconscious state is necessarily confounded by the presence of the drug used to induce the unconsciousness (G. Tononi and C. Koch, Ann N Y Acad Sci 1124, 239 (March, 2008)). Therefore, to identify the minimal neural correlates of consciousness during anesthesia, some method must be utilized to enable the brain activity patterns associated with the main effect of the drug to be disentangled from those associated with conscious information processing.

Here we solved this problem in two distinct ways and reveal brain activity patterns associated with the human conscious state and the voluntary intention to move in response to a command. First, we used neuroimaging pattern analysis software to visualize brain activity changes caused by various concentrations of two anesthetic agents during deep sedation, induction and emergence from the anesthetic state and separated the drug-related patterns of change from those associated with return of consciousness (ROC) following anesthesia. Second, we visualized the brain activity patterns that occur when conscious state is restored even when subjects' anesthetic states are held at the constant level.

In this study we have shown that the return of responsiveness following anesthesia requires activation of the brain stem, thalamus and the ACC as well as restored functional connectivity between the ACC and the parietal motor intention areas.

### Materials and Methods

### Participants

Twenty volunteers (healthy, right-handed, 19 to 28 years old males) participated in this open, non-randomized study approved by the local ethics committee and the National Agency for Medicines. All subjects gave written informed consent. None of the subjects had a history of psychiatric disorder, somatic illness, substance abuse, drug allergies, or ongoing medications and all subjects underwent a detailed pre-study physical examination. Subjects refrained from using alcohol or any medication for 48 hours before the study and they fasted overnight. No pre-medication was administered prior to study drug administration.

### Anesthesia Sessions

Subjects participated in two separate anesthesia sessions approximately one month apart. The first session was used to establish each subject's individual dose-response characteristics. The second session was used for neuroimaging. Subjects were anesthetized in a similar fashion on the two separate occasions. For each session, the subjects were prepared for the administration of a general anesthetic. In the first session, the individual concentrations for LOC were determined. Subjects were divided into two groups to receive either dexmedetomidine (n=10) or propofol (n=10) intravenous anesthesia via computer-controlled infusion. Once assigned to a particular anesthetic agent, that subject then again received that anesthetic during the neuroimaging session. Each infusion was started at a low rate and was then increased in a step-wise manner at 10 min intervals until LOC was detected. Consciousness was tested at 5 min intervals by asking the subjects to open their eyes. LOC was defined as being unresponsive to this request.

Dexmedetomidine (Precedex 100 μg/ml, Orion, 02200 Espoo, Finland) was administered intravenously using a target controlled infusion (TCI) scheme aiming at escalating pseudo steady-state plasma concentrations every 10 min. A Harvard 22 syringe pump (Harvard Apparatus, South Natick, Mass.) connected to a portable computer running STANPUMP software (Steven L. Schafer, M.D., Columbia University Medical Center, 622 W 168th St, PH 5-505 New York, N.Y. 10032-3725, USA) and the pharmacokinetic parameters of Talke were used (P. Talke et al., Anesthesiology 99, 65 (2003)). In the first session, the infusion was started at a target (plasma) concentration of 1.0 ng/ml, followed first by a 0.5 ng/ml target concentration increase and then by subsequent 0.25 ng/ml increases thereafter (i.e. 1.0-1.5-1.75-2.0-2.25-etc. ng/ml) until LOC was achieved.

Propofol (Propofol Lipuro 10 mg/ml, B. Braun Melsungen AG, Pfieffewiesen, D-34212 Melsungen, Germany) was administrated with the same infusion system and scheme as dexmedetomidine, using the Marsh pharmacokinetic model (B. Marsh et al., Br J Anaesth 67, 412 (1991)). In the first session, the infusion was started at a target (plasma) concentration of 1.0 μg/ml, followed first by a 0.5 μg/ml target concentration increase and then subsequent 0.25 μg/ml increases thereafter (i.e. 1.0-1.5-1.75-2.0-2.25-etc. μg/ml) until LOC was achieved.

In the second (neuroimaging) session of the study, the computer controlled drug infusion was repeated for each subject by aiming at their individually determined drug concentrations 50, 75 and 100% of the LOC concentration. Consciousness was tested similarly to the first session. If the LOC concentration defined in the first session of the study was not sufficient to produce LOC in the second session, the drug infusion was continued and an additional 25% target concentration increment was added to the rate. Concentrations were adjusted at 10 min intervals with simultaneous imaging at each level until LOC was detected. In the dexmedetomidine group, 1 subject lost consciousness at 50% of the previously measured LOC concentration value, 1 at 75% of LOC, 3 at 100% of LOC and 5 at 125% of LOC. As an awake scan was the only scan preceding the 50%-of-LOC scan in the one subject, this participant's data were excluded from those analyses that required all 4 scan conditions. Additionally, one subject's data were unusable due to technical difficulties. In the propofol group, 2 subjects lost consciousness at 75% of LOC, 6 at 100% of LOC and 1 at 125% of LOC. Due to scanner malfunction, the scans of one subject in the propofol group could not be completed. The cerebral perfusion measurement immediately preceding the LOC scan was used as the sedation scan in the analysis.

After LOC was reached and neuroimaging completed at that level of anesthesia, those in the propofol infusion group had the infusion terminated and the subjects were allowed to emerge from anesthesia. During the emergence process, consciousness was tested at 1 min intervals until ROC was detected. ROC was defined as eye opening in response to the command to do so. In the dexmedetomidine group consciousness was restored for the ROC scan using the unique property of dexmedetomidine whereby gentle tactile stimulation and/or loud voice was sufficient to restore consciousness even during a continuous infusion of the drug. PET measurements were thus obtained during the four conditions: baseline, sedation, LOC and ROC for 17 subjects. With the dexmedetomidine group, an additional cerebral perfusion measurement was performed as subjects remained in the scanner and were left unstimulated. This allowed them to lose their consciousness for the second time (LOC-2), shortly after performing the ROC scan. One subject in this group did not lose consciousness as the stimulation was stopped and, thus, LOC-2 scan could not be performed.

### Positron Emission Tomography (PET) Scanning

A previously described PET procedure for measuring cerebral perfusion was applied (K. K. Kaisti et al., Anesthesiology 96, 1358 (June, 2002)). Scans were obtained using an ECAT EXACT HR+PET scanner (Siemens/CTI, Knoxville, Tenn., USA) and [¹⁵O]H₂O as a tracer for assessment of regional cerebral perfusion. All [¹⁵O]H₂O scan data were corrected for detector dead time, tracer decay, and measured tissue photon attenuation, and reconstructed into a 128×128 matrix using a 3D transaxial Hann filter with a 4.6-mm cutoff and an axial filter. The field-of-view was 30 cm, resulting in pixel size of 300 mm/128=2.34 mm. Photon attenuation in the tissue was taken into account according to transmission scans obtained using three external circulating ⁶⁸Ge rod sources. Individual magnetic resonance images were acquired for anatomical reference with a 1.5 Tesla scanner (Gyroscan Intera CV Nova Dual, Philips Medical Systems, Best, the Netherlands) in a separate session.

### Parametric Images and Preprocessing

The subject's [¹⁵O]H₂O images were computed into quantitative parametric cerebral perfusion images using tracer kinetic modeling as previously described (K. K. Kaisti et al., Anesthesiology 96, 1358 (June, 2002)). The images were then realigned, coregistered, and spatially normalized using the image processing routines implemented within the SPM software package (SPM-8; Wellcome Department of Cognitive Neurology, University College London, London, England. Available at: www.fil.ion.ucl.ac.uk/spm) running under Matlab 2009b (MathWorks, Inc., Natick, Mass.). All normalized PET images were proportionally scaled and smoothed using an isotropic gaussian kernel of 12 mm full-width at half-maximum.

### Data Analysis

Cerebral perfusion images were analyzed for overall patterns of condition-related regional perfusion changes using Partial Least Squares (PLS) software (http://wvvw.rotman-baycrest.on.ca/index.php?section=84). PLS is a multivariate analysis technique used to detect optimal covariation between brain voxel-values and the experimental design (F. H. Lin et al., Neuroimage 20, 625 (October, 2003); N.J. Lobaugh et al., Psychophysiology 38, 517 (May, 2001)). A mathematical description of the method has been given in detail elsewhere (A. R. McIntosh et al., Neuroimage 3, 143 (June, 1996); A. R. McIntosh and N.J. Lobaugh, Neuroimage 23 Suppl 1, S250 (2004)). Briefly, in the first step, a cross-covariation matrix was computed using the design matrix and a matrix containing all of the voxel-values of each subject across all conditions (i.e., the data matrix). In the next step, the created cross-covariation matrix was decomposed using singular value decomposition (SVD) resulting in a set of latent variables (LVs). Each LV (constructed from a Singular image and a Singular value) successively accounts for a smaller portion of the detected covariance pattern until all is counted for. A singular image displays the brain regions exhibiting the greatest covariance between the design and the neuroimaging data as saliences (both positive and negative) while a singular value represents the extent of the covariation. The individual brain scores are used to estimate how an individual subject's scan reflects the overall pattern represented in the singular image. These brain scores are obtained by multiplying the singular image with the individual raw images. For the primary PLS analysis the propofol and the dexmedetomidine groups were combined based on the four behavioral conditions: baseline, sedation, LOC, ROC. An additional PLS analysis was performed to reveal the findings occurring as the dexmedetomidine anesthetized subjects regained and then again lost their consciousness for the second time (LOC-2). Functional connectivity analysis was performed using seed PLS (A. R. McIntosh et al., J Neurosci 23, 6520 (Jul. 23, 2003)).

### Statistical Methods

The statistical significance of each LV is determined using permutation tests by randomly re-ordering the data matrix rows and calculating a new set of LVs using SVD for each re-ordering. At each permutation, the singular value of each new LV is compared to the singular value of the original LV. The original singular value is then assigned a probability based on the number of times a statistic from the permuted data exceeds this original value (A R McIntosh et al., 1996). In the present study, 1000 permutations were computed.

The reliability of the saliences identified in each singular image is assessed by a bootstrap estimation of the standard error (1000 iterations) which provides evidence of the stability of the findings, rather than simply demonstrating whether an effect exists (A R McIntosh and N J Lobaugh, 2004). This procedure involves randomly resampling subjects with replacement, and computing the standard error of the saliences after several bootstrap samples. A voxel salience whose value remains stable regardless of the sample chosen is considered reliable. Voxels with saliences greater than 3.3 times their standard error (corresponding to an approximate p<0.001 on a two-tailed normal distribution) were considered reliable. As saliences are identified in one single analytic step, correcting for multiple comparisons is not necessary in PLS. For localizations, all resulting coordinates are listed in Montreal Neurologic Institute (MNI) space in mm.

### Results

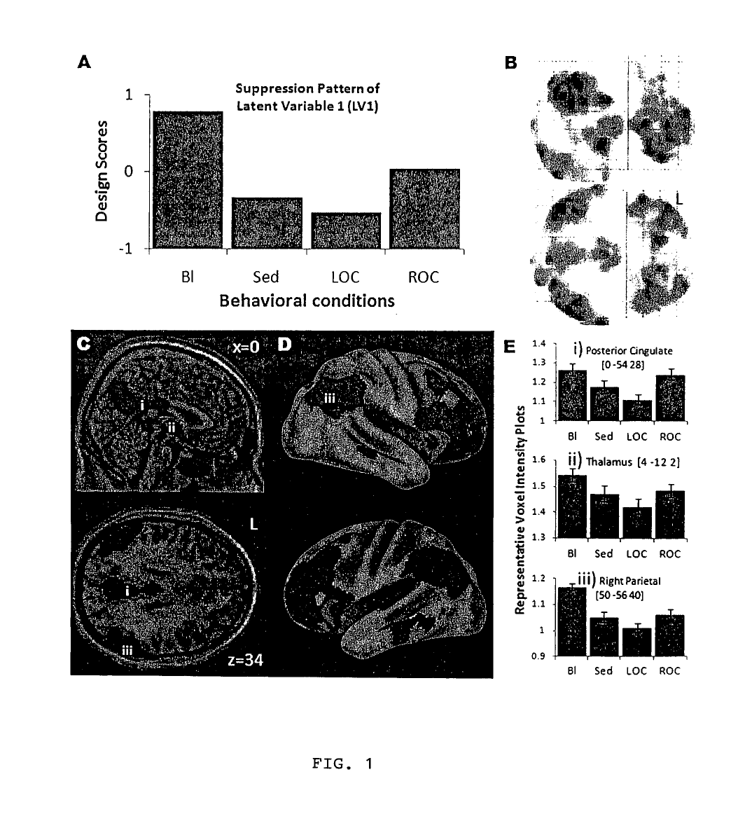

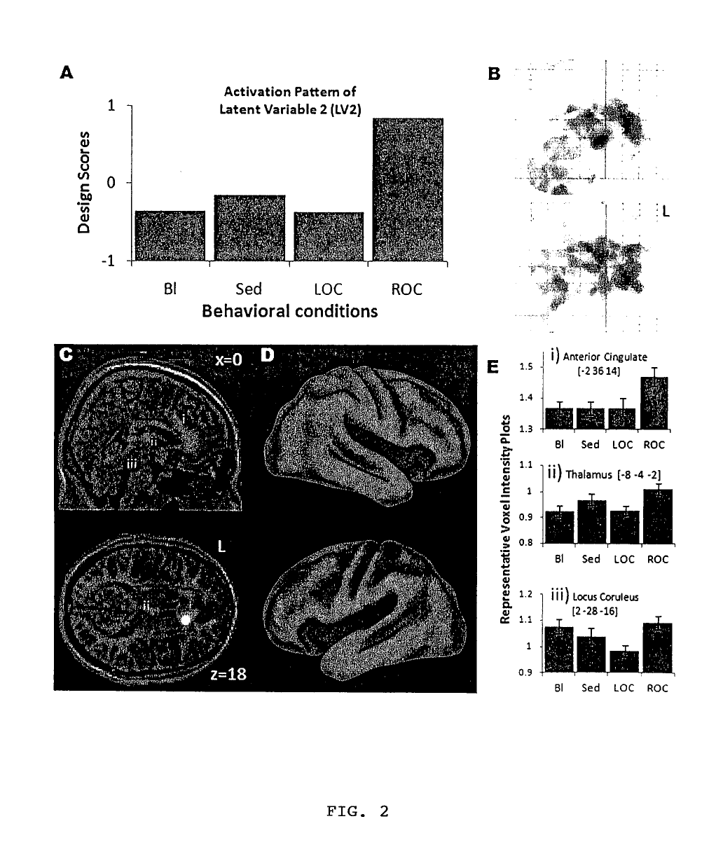

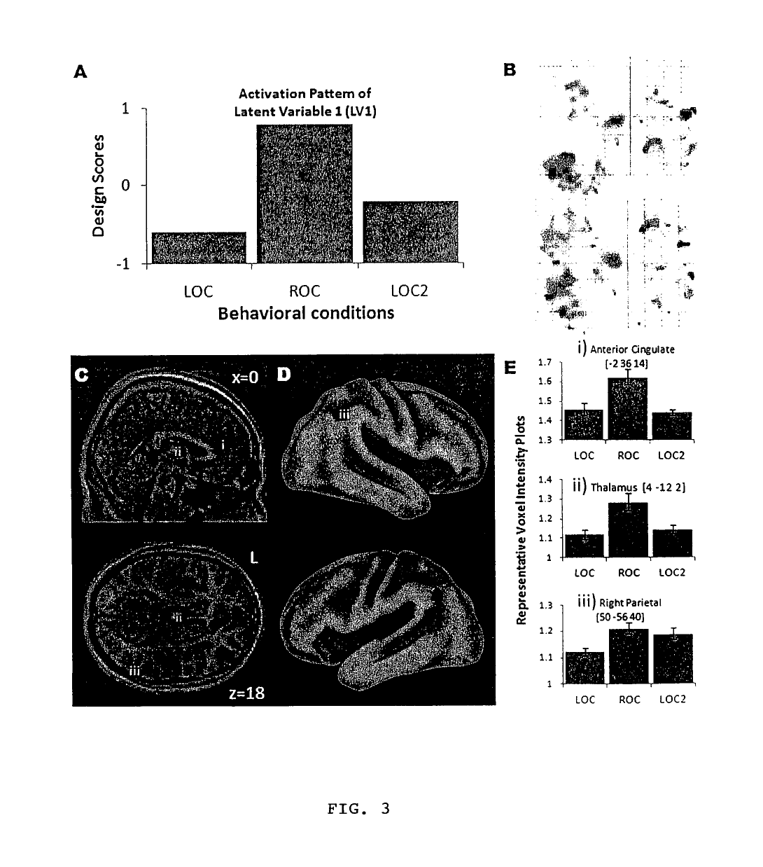

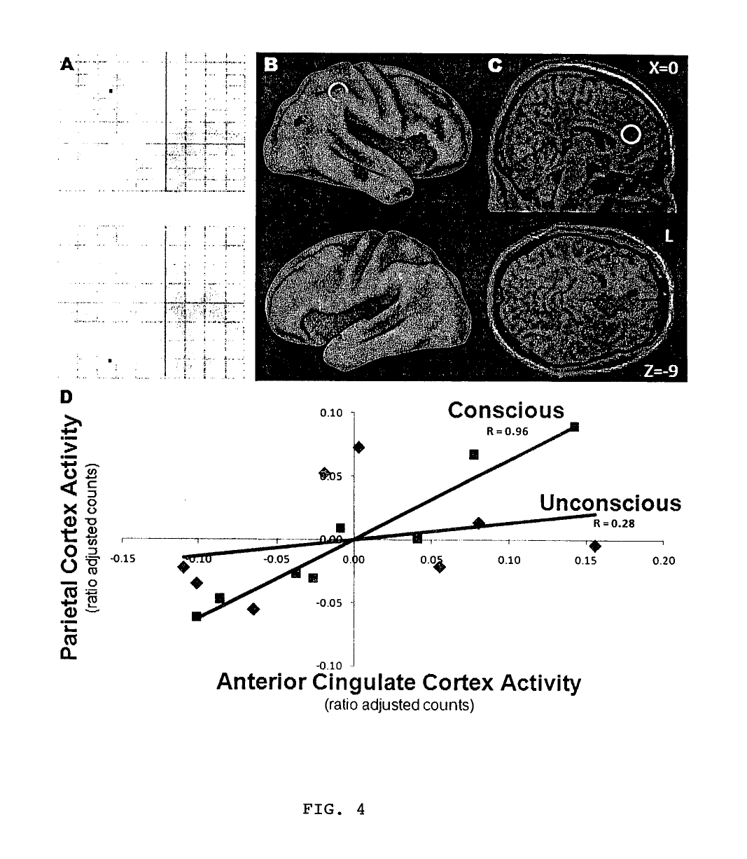

The main PLS analysis (FIGS. 1 and 2) yielded four LV's; LV1 (FIG. 1.), LV2 (FIG. 2.), LV3 and LV4. These respectively accounted for 67.7, 24.94, 7.34 and <1% of the cross block covariance detected in the data. LV3 was not statistically significant and thus not further analyzed, LV4 represents the residuals. The independent analysis of the individual anesthetic agents was performed in a similar manner and revealed similar LV structures. For the PLS analysis of the return of consciousness on dexmedetomidine the LOC, ROC and LOC-2 conditions yielded three LV's; LV1 (FIG. 3.), LV2 and LV3. These respectively accounted for 79.74, 20.26 and <1% of the cross block covariance. Only LV1 was significant. The activity in the lateral parietal cortex “intention to move” area was significantly related to activity in the ACC, but only when subjects were conscious and able to willfully respond (FIG. 4).

### Study II

### TMS-Stimulation of the Target Region(s) in the Individual's Brain

### Material and Methods

The effects of TMS on consciousness was assessed in two human subjects awake and anesthetized with escalating concentrations of dexmedetomidine. The two subjects in Study II had participated in Study I. It should be acknowledged that there may be agent dependent stimulation points that are more effective for one agent than another.

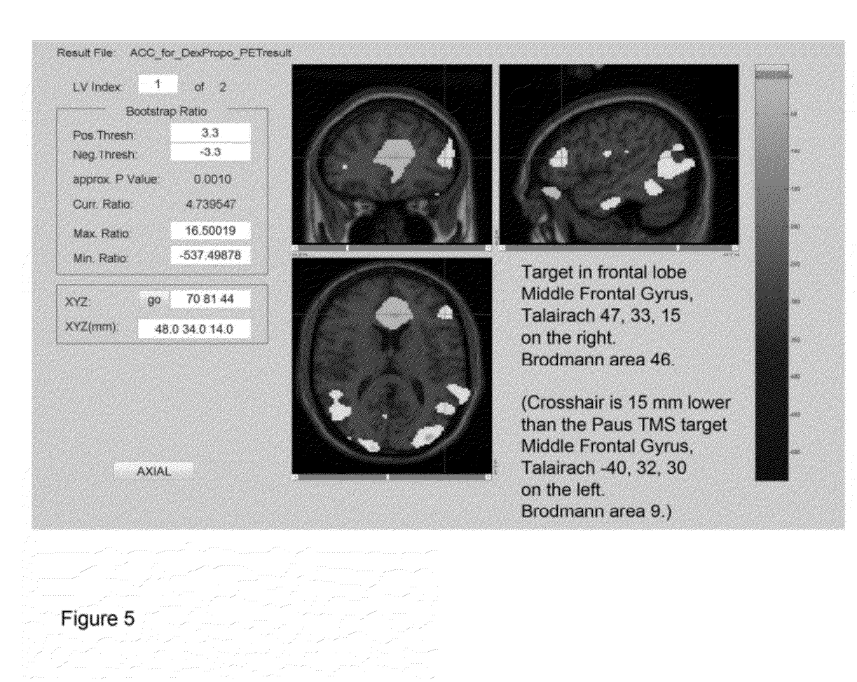

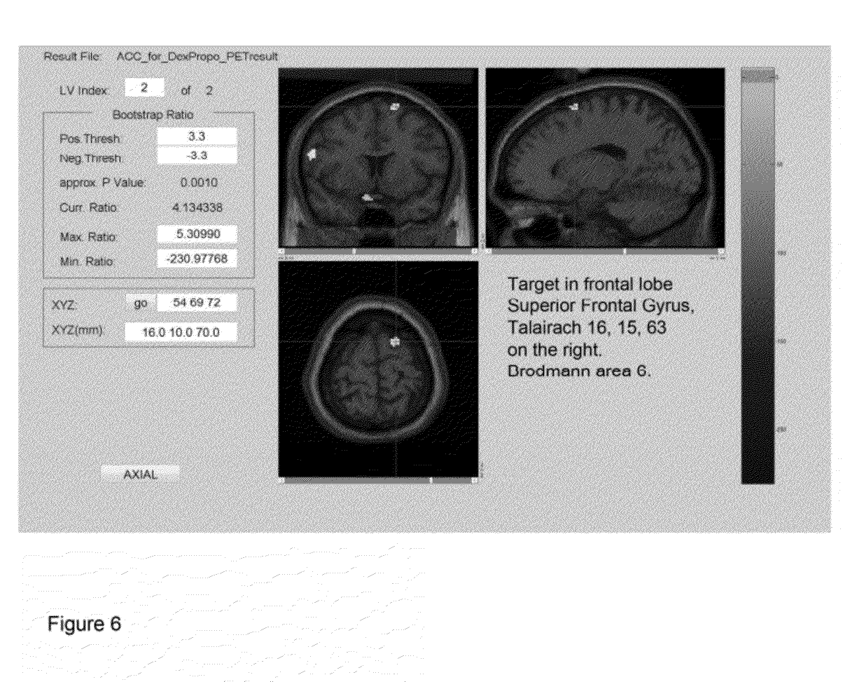

ACC stimulation/inhibition was accomplished indirectly through superficial cortical structures selected based on connectivity results from Study I and individual MRI DTI. Several cortical areas connected to ACC were found in PLS analyses. For example, Middle Frontal Gyrus (BA46, Talairach coordinates 47, 33, 15) was found to be highly negatively correlated with ACC activity in both the LOC and ROC conditions (FIG. 5). Superior Frontal Gyms (BA6, Talairach coordinates 16, 15, 63) represented an area, which was more correlated in LOC than ROC (FIG. 6).

MRI was performed using Siemens Magnetom Verio 3T scanner equipped with a 12 channel Head Matrix coil. Spin-echo EPI sequence was used for DTI acquisition (2.4 mm slice thickness, 128×128 acquisition matrix, 250 mm FOV, TR 7200 ms, TE 99 ms, b-values for diffusion weighting 0 and 1000 s/mm2, 64 gradient encoding directions, frequency selective saturation pulses were used for fat suppression).

DTI analyses (probabilistic tractography) were done using FDT tool of FSL Software Library (version 4.1.8) on Mac OS 10.6.8. ACC regions were used as a seed region and defined using highest point of activity on PET-images upon ROC observed in Study I as a centre for R 5 mm sphere. Goal regions were defined using coordinates provided as a centre of R 10 mm sphere. The goal regions were selected based on connectivity results from Study I and included, e.g., the Middle Frontal Gyms (BA46) and Superior Frontal Gyms (BA6). The total number of cortical goal regions analyzed was approximately 20 in both subjects. DTI results are provided as “number of tracts” and example images.

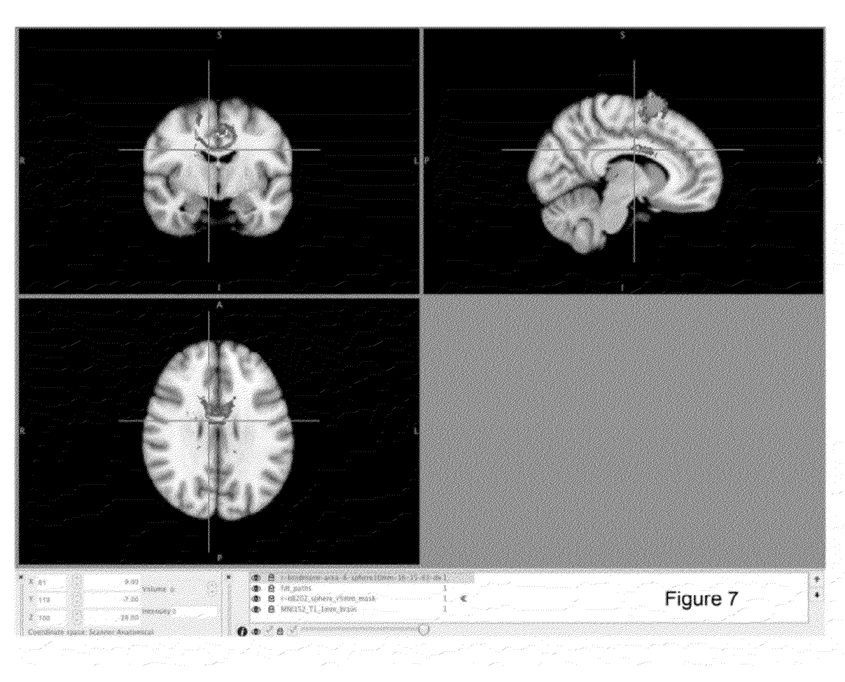

In general, especially the frontal (premotor) areas had anatomical connections to the ACC of which BA6 was clearly the strongest. The “number of tracts” between the ACC and BA6 on the right side were 4152 and 672, and between the ACC and BA46 on the right 32 and 35 in the two subjects, respectively. An example of tractography results from one subject showing a strong anatomical connection between the ACC and BA6 on the right hemisphere is shown in FIG. 7. Based on these results, BA6 was selected as the primary target for the TMS.

TMS was applied using the Navigated Brain Stimulation (NBS) device (Nexstim Oy, Helsinki, Finland) that enabled targeting of TMS pulses to specific cortical structures according to the individual head MRI. With high frequency serial pulses it is possible to activate and with low frequency serial pulses deactivate the target brain area. These effects are likely (or might possibly be related to) due to long-term potentiation (LTP) and long-term depression (LTD) effects of rTMS on the synaptic neural transmission, respectively.

First, the individual resting motor threshold (RMT) was determined in awake subjects by targeting single TMS pulses of different intensities to the representation area of the left thumb in the motor cortex and recording the evoked compound muscle action potential from the left opponens pollicis and abductor pollicis brevis muscles with surface electrodes and standard ENMG technique and device. Second, in the experiment, maximally 4000 repetitive pulses (four 1000 pulses series) using intensities from 90% to 110% of the individual RMT and frequencies from 1 Hz (inhibitory TMS) to 10 Hz (excitatory TMS) were given according the published rTMS safety guidelines to 3 target areas (BA6, BA46 and BA39/40, i.e. the “Desmurget area”) selected based on results from PET connectivity analyses and DTI imaging. The effects of inhibitory (<1 Hz) and excitatory rTMS (10 Hz) were assessed depending on the nature of correlation (positive or negative) between the ACC and the target (superficial) cortical structure and state of the subject (awake or unconscious). E.g., as the correlation was negative between BA46 and the ACC, excitatory rTMS to BA46 was supposed to inhibit and inhibitory rTMS to activate the ACC. The anesthesia sessions, after reaching LOC, were started with “placebo TMS” to BA6 by having a 10 cm long plastic cylinder between the coil and subject's head.

The level of sedation/anesthesia was assessed using the OAA/S scale, the Ramsey sedation score and the RASS before and after rTMS. Consciousness was tested by asking the subject to open his eyes as in Study I. In addition, subjective effects (vigilance, happiness, depression etc.) were measured before and after rTMS using a 100 mm visual analogue scale (VAS). TMS experiments were carried out at the Department of Clinical Neurophysiology, Turku University Hospital, using eXimia NBS System (Nexstim, Helsinki, Finland).

### Results

Both subjects got tired during inhibitory TMS to BA6. VAS for sedation was increased from 50 to 60 (subject A) and from 20 to 70 (subject B), respectively. The latter subject actually fell asleep during TMS to BA6, and sedation was increased also after excitatory TMS to BA46 (VAS for sedation increased from 10 to 40). Both subjects remained, however, arousable during the session.

In the anesthesia sessions performed on separate days, placebo TMS did not have any effects, i.e. the subjects remained unconscious. After approximately 500 excitatory rTMS pulses to BA6 subject B started to move and after 1000 pulses spontaneously tried to open his eyes. RASS increased from −5 to −2, Ramsay from 5 to 3 and OAA/S from 1 to 3. After stopping rTMS the subject lost his consciousness again. Then inhibitory rTMS was applied to BA46. After 1000 pulses, the subject opened his eyes upon request and answered to a question “How are you feeling?” with “I am feeling ok, but confused”. RASS, Ramsay and OAA/S scales increased from −5 to −2, from 5 to 3 and from 1 to 4, respectively. Excitatory rTMS to the “Desmurget area” did not have any effect. Subject A did not show, however, any signs of awakening during or after rTMS to BA6, BA46 or the “Desmurget area”.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. A method for altering an individual's level of consciousness by directing stimulation or inhibition to a CNS locus target region in the individual's brain that is responsible for consciousness.

2. The method according to claim 1, wherein the said target region is the ACC, cuneus, precuneus, prefrontal cortex, thalamus, hypothalamus, brainstem arousal areas such as the LC, or the parietal cortex, such as the intentional movement area.

3. The method according to claim 1, wherein the target region is a deeply located structure in the brain, such as ACC, and the stimulation is either
a direct excitatory or inhibitory stimulation of the deeply located structure, such as ACC, or
an excitatory or inhibitory stimulation of a superficial structure connected to the deeply located structure, to induce change in activity in the deeply located structure.

4. The method according to claim 3, wherein the superficial structure is a cortical area, such as the premotor cortex.

5. The method according to claim 3, wherein the superficial structure is sensory-motor/parietal cortex, in particular the parietal cortex intentional movement area.

6. The method according to claim 3, wherein several targets are stimulated and/or inhibited at the same time.

7. The method according to claim 3, wherein the superficial structure is superior fontal gyms or middle frontal gyrus.

8. The method according to claim 1, wherein the stimulation is TMS, optionally by using an H-coil or another specially designed coil in case deeply located regions shall be targeted.

9. The method according to claim 1, wherein the stimulation is audio stimulation, US, vestibular stimulation, nasal stimulation, focused laser beams, any form of electromagnetic radiation, light, heat and microwave to make a neuron fire.

10. The method according to claim 1, wherein the individual's consciousness is returned from a state of anesthesia, sedation, sleep, coma, vegetative state, and minimally conscious state.

11. The method according to claim 10, wherein the ROC is caused by inhibitory (<1 Hz) and/or excitatory TMS-stimulation (>5 Hz).

12. The method according to claim 1, wherein the level of consciousness is decreased.

13. The method according to claim 12, wherein the decrease of consciousness is caused by inhibitory (<1 Hz) and/or excitatory TMS-stimulation (>5 Hz).

14. The method according to claim 1, wherein the method additionally comprises determining of at least one target region in the individual's brain.

15. The method according to claim 14, wherein at least one target region in the individual's brain is determined by a non-functional method carried out in a separate session before initiating the stimulation.

16. The method according to claim 15, wherein the non-functional method is anatomical MRI or DTI MRI.

17. The method according to claim 14, wherein the step of determining includes obtaining a brain map of the brain using other methods.

18. The method of claim 17, wherein the brain map is obtained using at least one of fMRI and other MRI based methods, PET, SPECT, qEEG, MEG, US, NIRS and other optical methods, CT and EIT.
